(19) United States
(12) Patent Application Publication
(10) Pub. No.: US 2019/0201494 A1
(43) Pub. Date: Jul. 4, 2019

(54) COMPOSITIONS AND METHODS FOR TREATING NICOTINE WITHDRAWAL

(71) Applicant: STC UNM, Albuquerque, NM (US)

(72) Inventor: Ajna Hamidovic, Chicago, IL (US)

(21) Appl. No.: 16/331,691

(22) PCT Filed: Sep. 13, 2017
(86) PCT No.: PCT/US2017/051331

§ 371 Date: Mar. 8, 2019

Related U.S. Application Data
(60) Provisional application No. 62/393,924, filed on Sep. 13, 2016.

(51) Int. Cl. A61K 38/28; A61K 9/00; A61P 25/34

(52) U.S. Cl. CPC A61K 38/28; A61P 25/34; A61K 9/0043

(57) ABSTRACT

A method of treating a subject for acute nicotine withdrawal generally includes intranasally administering to the subject a dose of insulin effective to ameliorate at least one symptom or clinical sign of nicotine withdrawal. In some embodiments, the dose of insulin can be at least 45 IU and no more than 75 IU. In one particular embodiment, the dose can be 60 IU. In some embodiments, the dose can be an amount effective to restore at least a portion of a nicotine-withdrawal-associated decrease in saliva Cortisol level following a stress stimulus. In some embodiments, the dose can be an amount effective to decrease an urge to smoke a cigarette during cute withdrawal from nicotine.

### CROSS-REFERENCE TO RELATED APPLICATION

This application claims priority to U.S. Provisional Patent Application No. 62/393,924, filed Sep. 13, 2016, which is incorporated herein by reference.

### GOVERNMENT FUNDING

This invention was made with government support under R03 DA036054 and R03 DA038276 awarded by the National Institute on Drug Abuse. The government has certain rights in the invention.

### SUMMARY

This disclosure describes, in one aspect, a method of treating a subject for acute nicotine withdrawal. Generally, the method includes intranasally administering to the subject a dose of insulin effective to ameliorate at least one symptom or clinical sign of nicotine withdrawal.

In some embodiments, the dose of insulin can be at least 45 IU and no more than 75 IU. In one particular embodiment, the dose can be 60 IU.

In some embodiments, the dose can be an amount effective to restore at least a portion of a nicotine-withdrawal-associated decrease in saliva cortisol level following a stress stimulus.

In some embodiments, the dose can be an amount effective to decrease an urge to smoke a cigarette during acute withdrawal from nicotine.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Baseline salivary cortisol and subjective ratings. The measures were taken at the time of session check-in (8:30 AM) following overnight abstinence from smoking. Treatment groups did not differ in their (A) salivary cortisol (p=0.50), (B) nicotine cravings (p=0.19), (C) positive mood (p=0.97), and (D) negative mood (p=0.67).

FIG. 2. Nicotine craving ratings during the course of study session. Smoking urges decreased significantly with intranasal insulin compared to placebo (p≤0.05). The effect of intranasal insulin was detected following spray administration and lasted for 100 minutes through the stress task. “INSTRUCTIONS” and “SPEECH” on the graph below refer to the two parts of Trier Social Stress Test. “TREATMENT” refers to either insulin or placebo spray administration. Y-axis refers the predictive margins of the mixed model analyzing nicotine craving scores. X-axis reflects clock time (*p≤0.05; **p≤0.01).

FIG. 3. Graphic presentation of salivary cortisol levels results. Data are log-transformed to normalize distribution and results are presented as peak change from baseline. Intranasal insulin significantly increased peak cortisol levels following Trier Social Stress Test procedure (p≤0.001).

FIG. 4: Study blood measurements results. Treatment groups did not differ in their (A) insulin (p=0.88), (B) glucose (p=0.28), (C) c-peptide (p=0.23), and (D) non-esterified fatty acids (p=0.80) (all p values reflect main effect of treatment). Time was significant at p≤0.001 level for (C) c-peptide and (D) non-esterified fatty acids, but at p≤0.01 level for (B) insulin. Treatment×time interaction was not significant for any of the measures other than glucose (p≤0.05).

FIG. 5. Replication study nicotine craving ratings. The crossover study involved 36 hours of smoking abstinence with Questionnaire of Smoking Urges measurements taken on the morning of Day 2. Smoking urges decreased significantly with intranasal insulin compared to placebo (p≤0.05). Y-axis refers to the predictive margins of the mixed model analyzing nicotine craving scores. X-axis reflects clock time.

FIG. 6. Questionnaire of Smoking Urges—Brief Factor 1 (strong desire and intention to smoke with smoking perceived as rewarding). Intranasal insulin significantly decreased Factor 1 scores as indicated by a significant time×treatment interaction (p≤0.05). The effect of intranasal insulin was detected immediately following treatment administration and lasted for 100 minutes minimum (*p≤0.05; **p≤0.01).

FIG. 7. Questionnaire of Smoking Urges—Brief Factor 2 (anticipation of relief from negative affect with an urgent desire to smoke). There was no effect of intranasal insulin on Factor 2 scores as indicated by a non-significant time×treatment interaction (p=0.30).

FIG. 8. Negative mood ratings as measured by Positive and Negative Affect Schedule (PANAS). Intranasal insulin had no effect on negative mood rating as indicated by a non-significant time×treatment interaction (p=0.84).

FIG. 9. Positive mood ratings as measured by Positive and Negative Affect Schedule (PANAS). Intranasal insulin had no effect on negative mood rating as indicated by a non-significant time×treatment interaction (p=0.61).

FIG. 10. Replication study Questionnaire of Smoking Urges—Brief Factor 1 (strong desire and intention to smoke with smoking perceived as rewarding). In comparison to placebo, intranasal insulin significantly decreased Factor 1 scores (p≤0.05).

FIG. 11. Replication study Questionnaire of Smoking Urges—Brief Factor 2 (anticipation of relief from negative affect with an urgent desire to smoke). There was no effect of intranasal insulin on Factor 2 scores as indicated by a non-significant effect of treatment (p=0.18).

FIG. 12. Subjective ratings following spray administration. Treatment was administered as one spray in each nostril every three minutes for the total of six sprays. After each round of sprays, on a scale of 0-10, research participants rated (A)“pain”, (B)“burn” and (C)“taste”. In the mixed model analyses, treatment×spray number interactions were not significant for any of the three ratings. Main effect of treatment was significant for “burn” and “taste” (p≤0.05 for both), but not for “pain” (p=0.24).

### DETAILED DESCRIPTION OF ILLUSTRATIVE EMBODIMENTS

This disclosure describes methods of treating the stress response occurring in the early phase of smoking abstinence. The methods generally include administering insulin intranasally to a subject. Insulin modulation significantly influences smoking behavior. A locus in insulin degrading enzyme was significantly associated with serum cotinine levels in both African and European American cohorts. Indeed, the role of neuropeptides (such as insulin) has been a growing area of addiction research, as they relate specifically to stress-induced drug reinstatement. Finally, nicotine and insulin have some overlapping cellular mechanisms and anatomical targets. Thus, during cessation of smoking, insulin can replace actions of nicotine on critical behavioral mechanisms. For example, both nicotine and insulin activate pro-opiomelanocortin (POMC) neurons in the arcuate nucleus of the hypothalamus through α3β4 and insulin receptors, respectively. The resulting increase in the second order neuron melanocortin 4 (MC4) receptor signaling provides inhibitory tone that restrains food intake and weight gain. Additionally, as is the case with nicotine, insulin significantly enhances striatal dopamine release by activating intercholinergic neurons, thereby signaling reward.

The intranasal route of administration to humans increases CSF insulin levels without changing peripheral insulin levels. The efficacy of intranasal insulin therapy is established in the report of a randomized, placebo-controlled, laboratory-based study with overnight abstinent smokers, testing the hypothesis that intranasal insulin (60 IU) decreases nicotine cravings and normalizes maladaptive allostatic overload without effecting peripheral glycemic measures. The initial results are reinforced by a second study establishing that intranasal insulin (60 IU) reduces nicotine cravings by providing results from our separate randomized, placebo-controlled crossover study over thirty-six hours of smoking abstinence.

Four hundred ninety-nine subjects were telephone screened to participate in the study. Fifty-six entered the randomization schedule and completed session 1 assessing cognitive measures. Fifty-one subjects were eligible for continuation to session 2. Following exclusion, the total number of included in the analysis was 37 (Table 1) including 16 randomized to intranasal insulin and 21 randomized to placebo. All patients smoked at least 10 cigarettes per day, and were, on average, moderately dependent as measured by Fagerstrom Test of Nicotine Dependence. All study participants were in the normal to overweight Body Mass Index category. Subjects did not differ on any of the demographic or physiological variables presented in Table 1.

TABLE 1
Demographic and Physiological Characteristics of Study Participants

| | Intranasal Insulin | Placebo |
|---|---|---|
| Characteristic | (N = 16) | (N = 21) |
| Age, mean (SD) | 35.75 (13.23) | 40.19 (13.74) |
| Gender (male) (%) | 81.25 | 66.67 |
| Race (white) (%) | 90.00 | 100.00 |
| Ethnicity (%) | 12.50 | 38.10 |
| Education (%) | | |
| <High school | 12.50 | 0.00 |
| High school graduate | 25.00 | 33.33 |
| Some college | 43.75 | 52.38 |
| College graduate | 6.25 | 14.29 |
| Graduate school Degree | 12.50 | 0.00 |
| Employed full time (%) | 37.50 | 38.10 |
| Body Mass Index, mean (SD) | 25.53 (3.62) | 25.29 (3.37) |
| Fagerstrom Test for Nicotine | 4.21 (2.86) | 5.42 (1.69) |
| Dependence, mean (SD) | | |
| Smoking quantity (cigarettes | 18.25 (13.42) | 18.28 (4.10) |
| per day), mean (SD) | | |
| Age started smoking, mean (SD) | 16.93 (3.31) | 16.25 (3.12) |
| Alcohol (within the last 30 days) (%) | 71.43 | 47.37 |
| Cannabis (more than 12 months ago) | 57.14 | 53.85 |
| (%) | | |
| Substance Abuse (ever) (%) | | |
| Cocaine | 50.00 | 42.11 |
| Heroin | 6.25 | 10.00 |
| Hallucinogens | 56.25 | 50.00 |
| Pain Relievers | 31.25 | 10.00 |
| Methamphetamine | 18.75 | 35.00 |

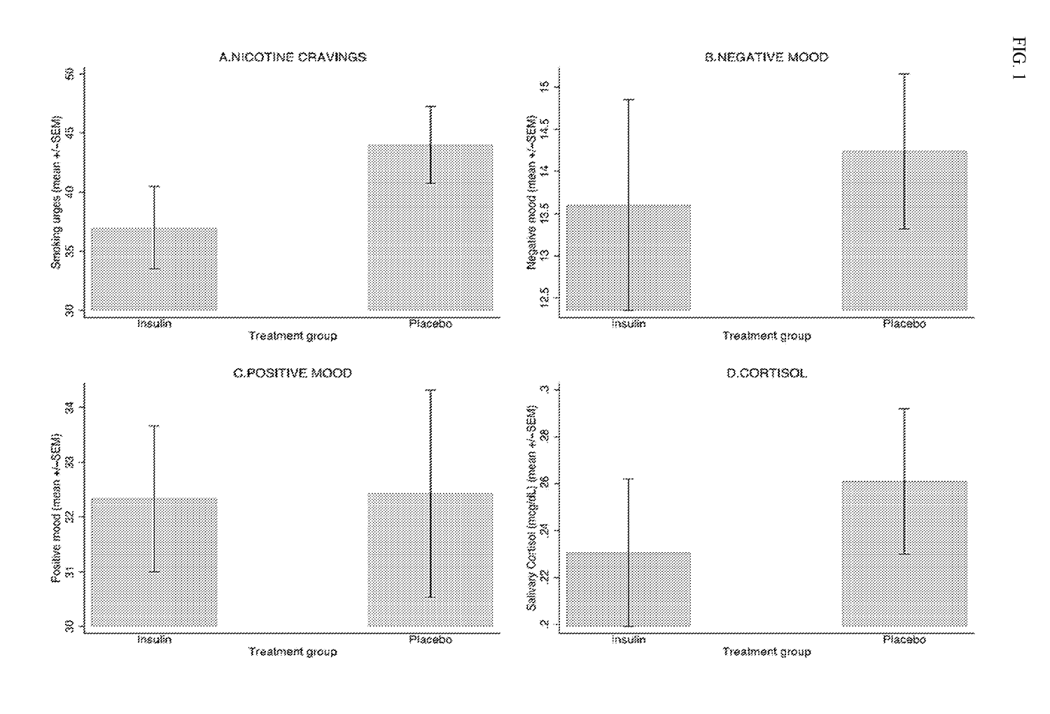

FIG. 1 displays baseline (i.e., 08:30) values of the two treatment groups. Treatment groups did not differ on their morning ratings of nicotine cravings (Mean difference=−6.42, SE=4.79, p=0.19). Additionally, the groups were not different with respect to their ratings of positive (Mean difference=−0.09, SE=2.51, p=0.97) and negative mood (Mean difference=−0.63, SE=1.51, p=0.67). Finally, treatment groups did not differ in their salivary cortisol levels (Mean difference=−0.03, SE=0.044, p=0.50).

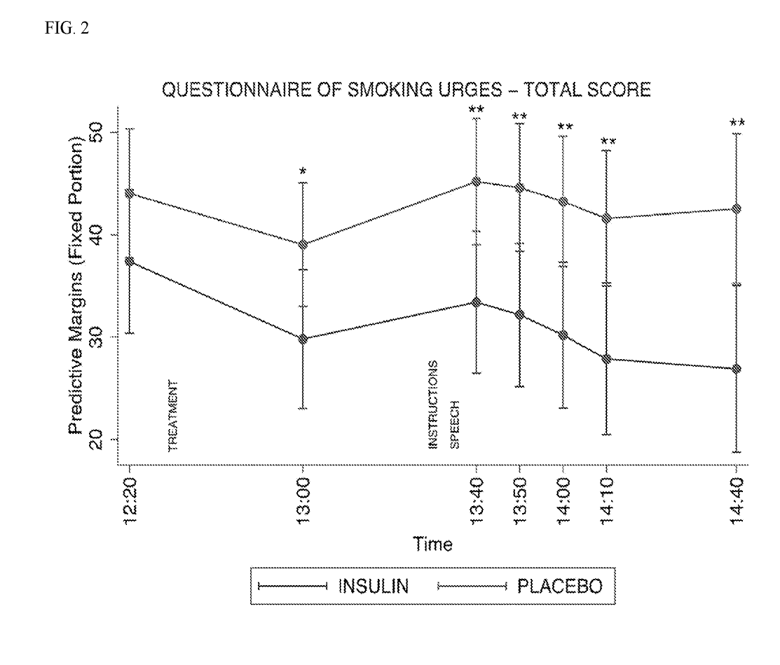

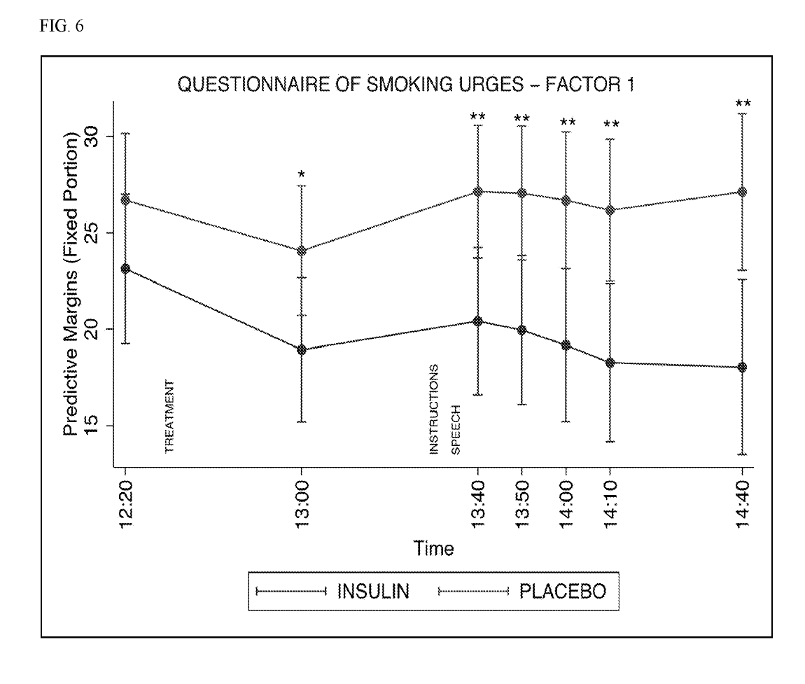

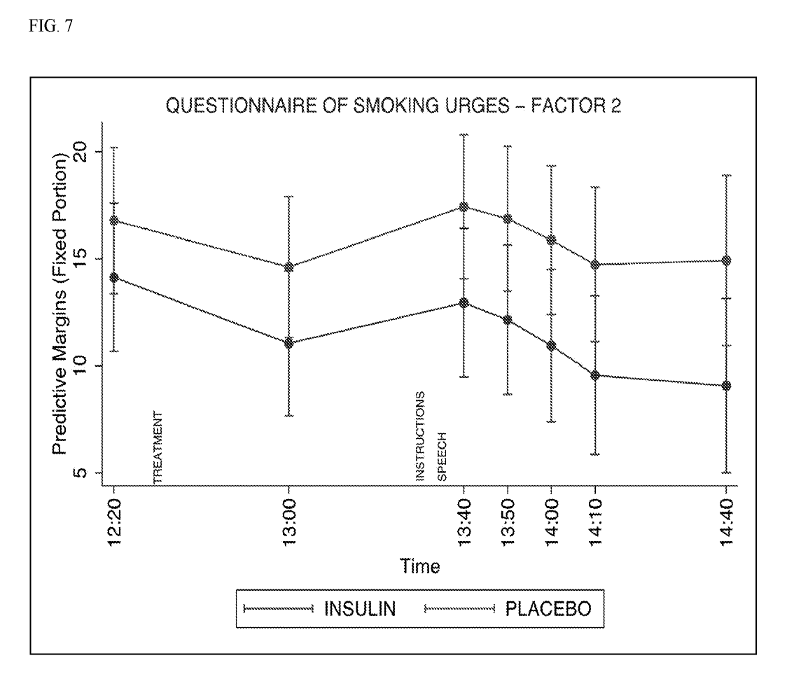

Smoking urges decreased significantly with intranasal insulin compared to placebo, as indicated by a significant treatment×time interaction (b=0.064, SE(b)=0.032, z=1.93, p≤0.05) (FIG. 2). Post hoc tests comparing treatment groups at each time point detected that the effect of intranasal insulin begun at 13:00 (i.e., at the first time point after spray administration) and lasted for 100 minutes (post hoc comparisons significance values at individual times: 13:00 (p≤0.05); 13:40, 13:50, 14:00, 14:10, and 14:40 (p≤0.01)). Cravings significantly changed over time (b=−0.87, SE(b)=0.15, z=−5.69, p≤0.001) while main effect of treatment was not significant (b=6.66, SE(b)=4.81, z=1.39, p=0.16). Sub-analyses, evaluating whether reductions in overall smoking urges were driven by Factor 1 or Factor 2 sub-scale revealed that the overall decrease in smoking urges was driven by intranasal insulin's effect on Factor 1 (treatment×time interaction: b=0.039, SE(b)=0.018, z=2.15, p≤0.05; main effect of time: b=−0.41, SE(b)=0.08, z=−4.91, p≤0.001; main effect of treatment: b=3.55, SE(b)=2.61, z=1.36, p=0.17), not Factor 2 scale (treatment×time interaction: b=0.02, SE(b)=0.01, z=1.48, p=0.14; main effect of time: b=−0.44, SE(b)=0.08, z=−5.21, p≤0.001; main effect of treatment: b=3.76, SE(b)=2.37, z=1.58, p=0.11). (FIG. 6 and FIG. 7).

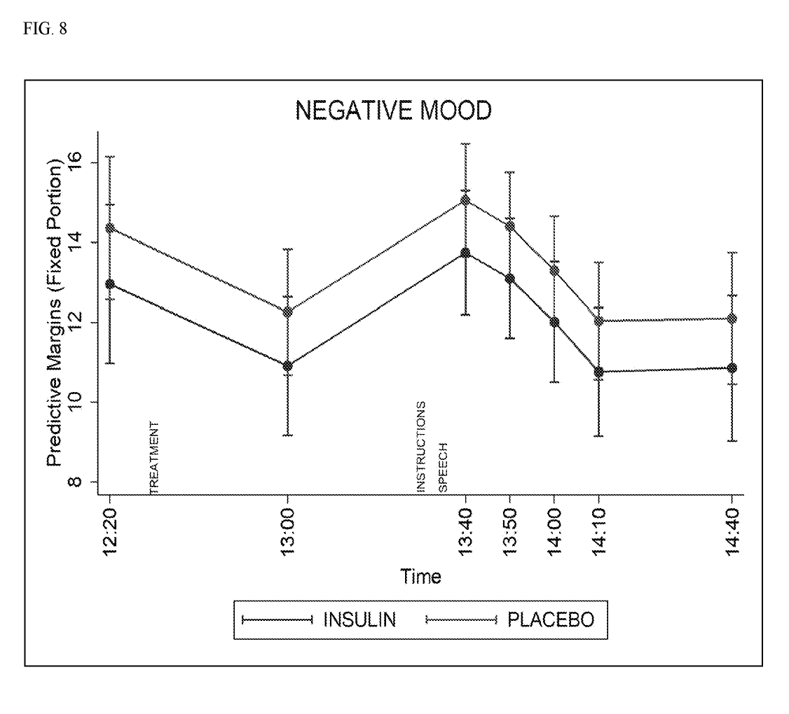

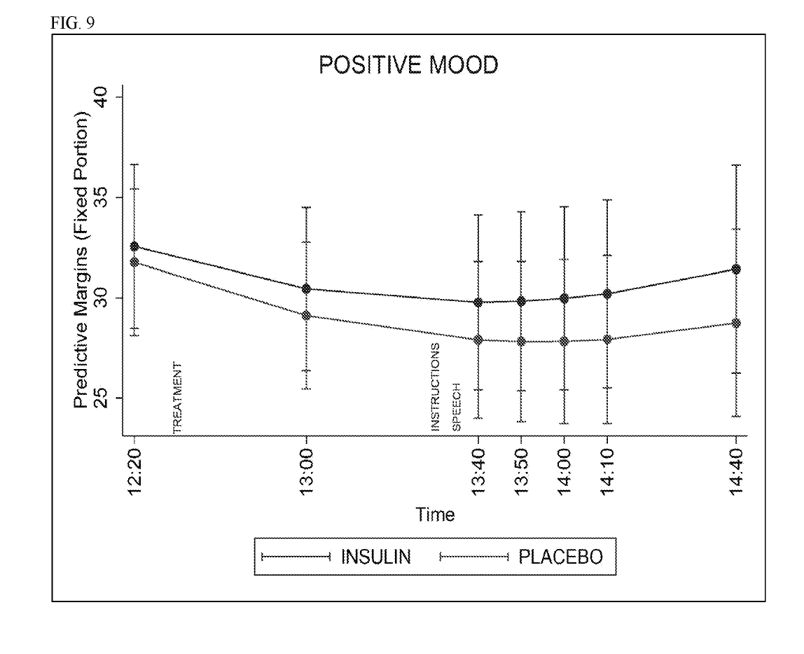

Ratings of negative mood significantly changed over time (b=−0.43, SE(b)=0.06, z=−6.41, p≤0.001). However, the interaction was not significant (b=0.001, SE(b)=0.01, z=0.10, p=0.92) and the effect of treatment was not significant (b=1.38, SE(b)=1.30, z=1.07, p=0.28). (FIG. 8). Similarly, rating of positive mood significantly changed over time (b=−0.07, SE(b)=0.02, z=−3.68, p≤0.001), but neither the interaction (b=−0.01, SE(b)=0.017, z=−0.97, p=0.33) nor treatment (b=−0.47, SE(b)=2.47, z=−0.19, p=0.84) were significant. (FIG. 9).

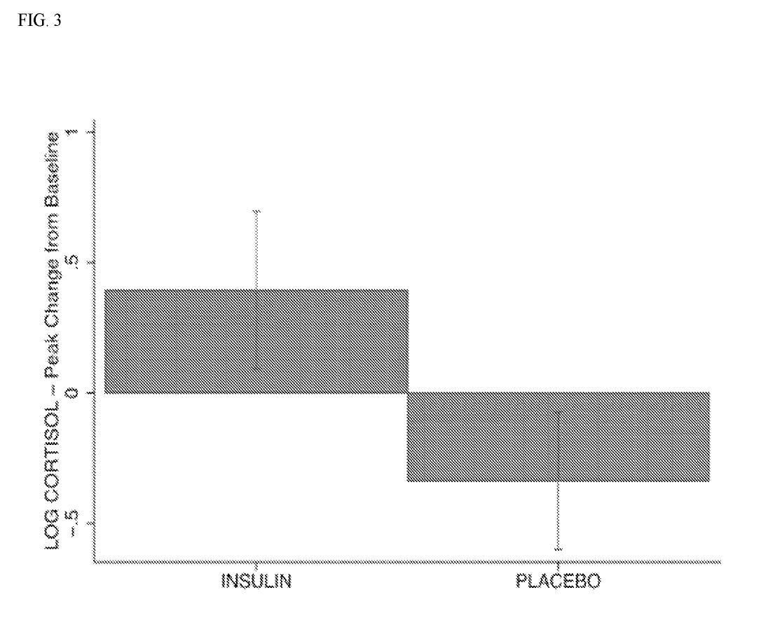

Intranasal insulin significantly increased peak cortisol levels following stress procedure F_{(1,35)}=12.78, p≤0.001. FIG. 3 shows a graph of log-transformed cortisol levels according to treatment group.

Trier Social Stress Test significantly increased systolic blood pressure over time (b=3.29, SE(b)=4.18, z=1.55, p≤0.001). Main effect of treatment was not significant (b=6.48, SE(b)=4.18, z=1.55, p=o.12). Treatment×time interaction was not significant (b=−0.46, SE(b)=0.32, z=−1.46, p=0.14). Diastolic blood pressure significantly increased over time (b=2.64, SE(b)=0.56, z=−1.92, p≤0.001). Main effect of treatment was not significant (b=4.64, SE(b)=2.58, z=1.80, p=0.07). The interaction was significant (b=−0.52, SE(b)=0.27, z=−1.92, p≤0.05). Trier Social Stress Test talk significantly increased heart rate over time (b=6.08, SE(b)=4.23, z=2.24, p≤0.05), however treatment groups did not differ (treatment: b=0.01, SE(b)=4.23, z=0.00, p=0.99; treatment×time: b=−0.81, SE(b)=1.03, z=−0.79, p=0.42).

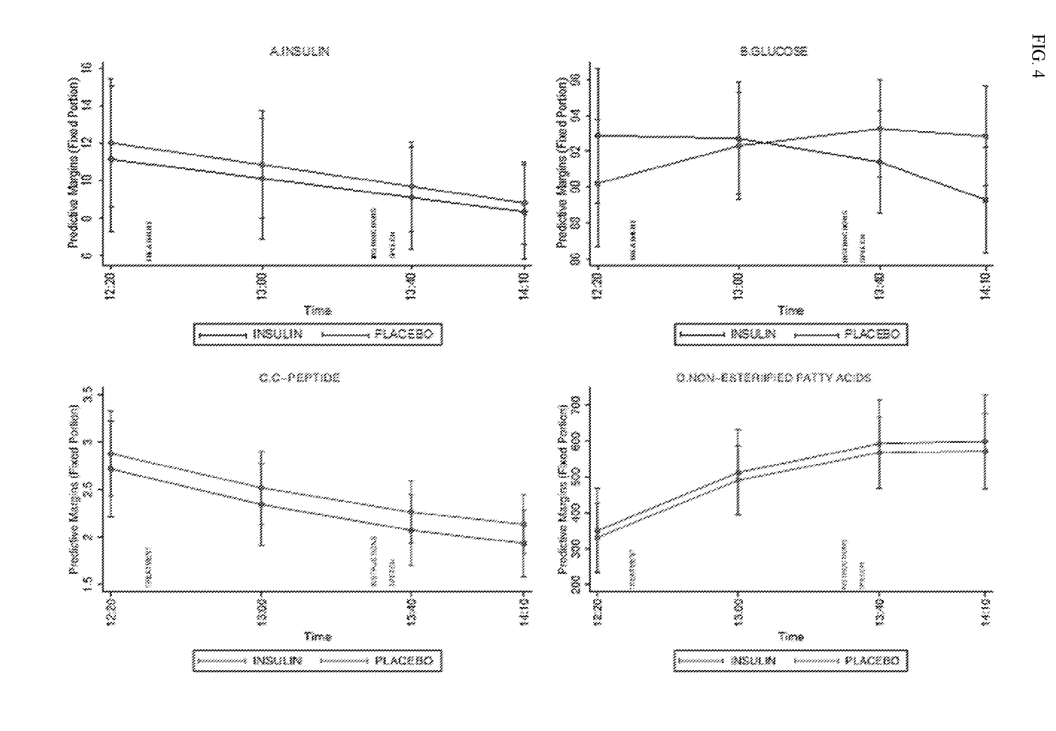

FIG. 4 shows results of insulin, glucose, C-peptide, and non-esterified fatty acids analyses. Insulin levels significantly decreased over time (b=−0.029, SE(b)=0.011, z=−2.62, p≤0.01) with no detection of treatment effects as evidenced by a non-significant interaction finding (b=−0.005, SE(b)=0.01, z=−0.37, p=0.71) as well as a non-significant main effect of treatment (b=0.42, SE(b)=2.82, z=0.15, p=0.88) finding. Although a treatment×time interaction was detected for glucose (b=0.05, SE(b)=0.02, z=2.60, p≤0.01) subjects remained well within euglycemic range. There was no evidence of an effect on glucose for either time (b=0.015, SE(b)=0.022, z=0.53, p=0.59) or treatment (b=−2.72, SE(b)=2.55, z=−1.06, p=0.28). C-peptide levels significantly decreased over time (b=−0.01, SE(b)=0.002, z=−4.91, p≤0.001) with no detection of treatment effect as evidenced by a non-significant interaction finding (b=0.0006, SE(b)=0.001, z=0.35, p=0.72) as well as a non-significant main effect of treatment (b=0.38, SE(b)=0.32, z=1.18, p=0.23) finding. There was evidence of non-esterified fatty acids increase over time (b=4.8, SE(b)=0.74, z=6.44, p≤0.001) which occurred for both treatment groups (main effect of treatment: b=−18.22, SE(b)=76.39, z=−0.24, p=0.80; time×treatment interaction: b=−0.05, SE(b)=0.44, z=0.13, p=0.89).

### Replication Study

Following telephone screening of three hundred and fifty-nine individuals, twenty-six were randomized for participation in the study. Of those, nineteen completed the entire cross-over schedule with 10 participants receiving insulin first and nine participants receiving placebo first. Table 2 summarizes the demographic and physiological characteristics of subjects completing the study. All patients smoked at least 10 cigarettes per day, and were, on average, moderately dependent as measured by Fagerstrom Test of Nicotine Dependence. All study participants were in the normal to overweight Body Mass Index category, with ˜20% body fat. Subjects receiving intranasal insulin vs. placebo first did not differ on any of the demographic or physiological variables presented in Table 2.

TABLE 2
Demographic and Physiological Characteristics
of Replication Study Participants

| | Intranasal Insulin^{a} | | Placebo^{b} | |
|---|---|---|---|---|
| | (N = 10) | | (N = 9) | |
| Characteristic | | | | |
| Age, mean (SD) | 29.30 | (7.14) | 38.33 | (12.82) |
| Male, gender, No. (%) | 8 | (80.00%) | 7 | (77.78%) |
| Race, No (%)^{d} | | | | |
| Black | 0 | (0%) | 2 | (22.00%) |
| White | 9 | (90.00%) | 4 | (40.00%) |
| Other/Multiple | 1 | (10.00%) | 2 | (25.00%) |
| Hispanic ethnicity, No. (%) | 0 | (0%) | 2 | (22.22%) |
| Education, No (%) | | | | |
| High school graduate | 3 | (30%) | 2 | (22.22%) |
| Some college | 6 | (60.00%) | 3 | (33.33%) |
| ≥4 Years of College | 1 | (10%) | 4 | (44.44%) |
| Unemployed, No (%) | 2 | (20%) | 5 | (55.55%) |
| Fagerstrom Test for Nicotine | 4.10 | (1.44) | 4.55 | (1.42) |
| Dependence, mean (SD) | | | | |
| Smoking quantity (cigarettes per | 14.70 | (4.19) | 15.77 | (9.23) |
| day), mean (SD) | | | | |
| Age started smoking, mean (SD) | 14.60 | (3.40) | 14.33 | (6.02) |
| Body Mass Index, mean (SD) | 24.80 | (3.40)^{c} | 24.87 | (2.68) |
| Glucose (mg/dL, Fasting), mean | 92.00 | (6.87) | 94.61 | (8.17) |
| (SD) | | | | |

^{a}Subjects randomly assigned to receive intranasal insulin first
^{b}Subjects randomly assigned to receive placebo first
^{c}Missing data for 1 subjects randomly assigned to receive intranasal insulin first
^{d}Missing data for 1 subjects randomly assigned to receive placebo first

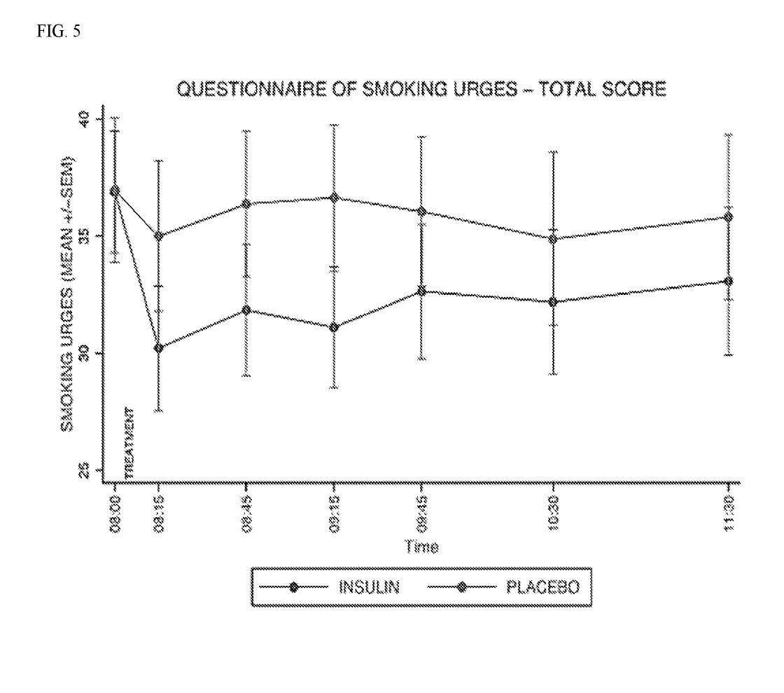

Smoking urges decreased significantly with intranasal insulin compared to placebo (b=3.67, SE(b)=1.91, z=1.92, p≤0.05) (FIG. 5). There was no evidence of effects related to the sequence of spray administration (b=3.27, SE(b)=4.90, z=0.67, p=0.50). Time was not significant (b=o.001 SE(b)=0.006, z=0.20, p=0.84). Time×treatment interaction was not significant (b=−o.003 SE(b)=0.007, z=−0.39, p=0.69).

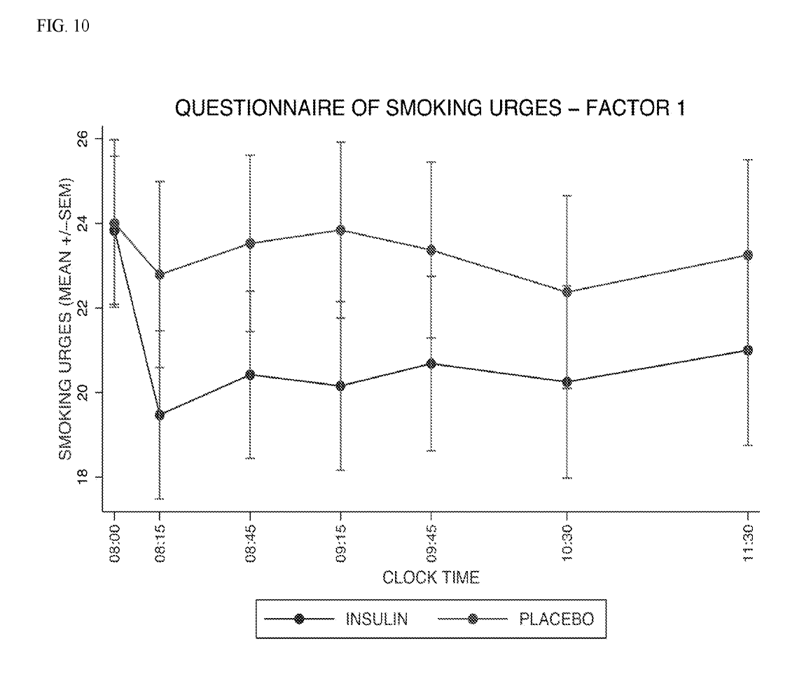

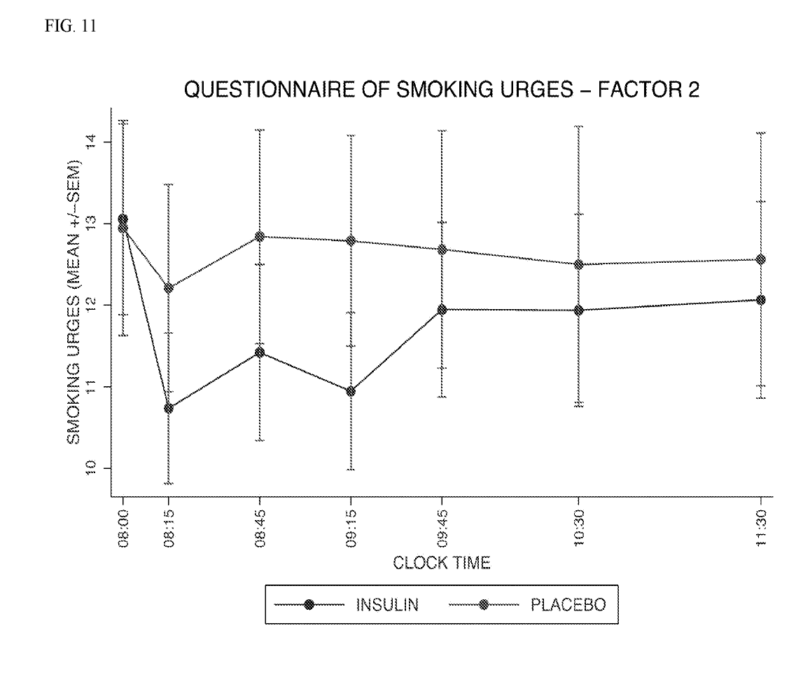

Sub-analyses, evaluating whether reductions in overall smoking urges were driven by Factor 1 or Factor 2 sub-scale revealed that the effect was driven by intranasal insulin's effect on Factor 1 (b=2.49, SE(b)=1.15, z=2.16, p≤0.05), not Factor 2 scale (b=1.16, SE(b)=0.87, z=1.34, p=0.18) (FIG. 10 and FIG. 11).

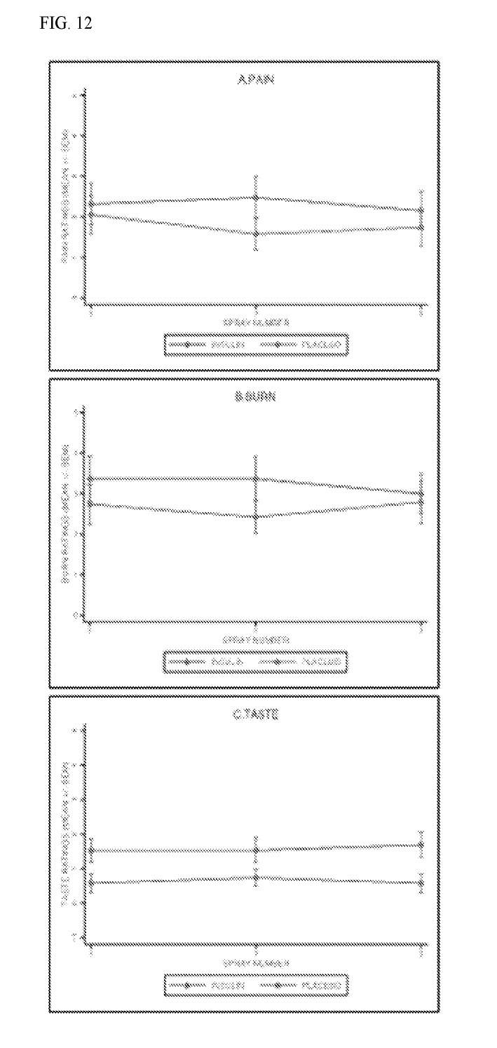

In the crossover analysis, intranasal insulin and placebo had similar effects on subjective spray ratings of “pain”. No group differences were detected for ratings of pain (treatment: b=−0.36, SE(b)=0.31, z=−1.17, p=0.24; spray number: −0.07, SE(b)=0.17, z=−0.45, p=0.65; treatment×spray number b=−0.07, SE(b)=0.16, z=−0.47, p=0.63; sequence b=0.79, SE(b)=0.77, z=1.02, p=0.31). There was an effect of treatment on the rating of “burn” (treatment: b=−1.01, SE(b)=0.43, z=−2.34, p≤0.05), which remained the same over the three sprays as the interaction with treatment was not statistically significant (b=0.21, SE(b)=0.17, z=1.23, p=0.22). In the model, spray number (b=−0.18, SE(b)=0.15, z=−1.17, p=0.24) and sequence (b=0.44, SE(b)=0.90, z=0.49, p=0.62) were not statistically significant. Similarly, on the measure of subjective ratings of burn, there was an effect of treatment on the rating of “taste” (treatment: b=−0.78, SE(b)=0.32, z=−2.44, p≤0.05), which remained over the three sprays as the interaction with treatment was not statistically significant (b=−0.07, SE(b)=0.10, z=−0.75, p=0.45). In the model, spray number (b=0.07, SE(b)=0.32, z=−2.44, p=0.29) and sequence (b=0.60, SE(b)=0.49, z=1.22, p=0.22) were not statistically significant. Subjective spray ratings are presented in FIG. 12.

Thus, intranasal insulin 60 IU was associated with a significant reduction of nicotine cravings compared to placebo. This finding was replicated in a separate study involving assessment of morning nicotine cravings following 36 hours of smoking abstinence. Greater reduction in nicotine cravings was present for one hundred minutes through psychosocial stress, although the effect may have extended beyond our pre-specified measurement period. This finding provides evidence that insulin disrupts neuro-modulatory mechanisms of rewarding effects of nicotine. Whereas intranasal insulin decreased nicotine cravings, it increased (i.e., restored) the blunted cortisol response to stress. As heightened stress-induced cravings and the severity of blunted response to stress predict worse smoking cessation outcomes, insulin's ability to reverse the unfavorable stress-induced state during acute (i.e., up to 36 hours) abstinence makes insulin a reasonable candidate for further clinical investigation.

Centrally, insulin tightly modulates dopamine firing via glutamatergic and cholinergic mechanisms in the insulin receptor abundant ventral striatum. Excitatory inputs onto ventral tegmental area undergo long-term depression (LTD) in the presence of insulin. The phenomenon requires presynaptic inhibition of glutamate release, and results in reduced food anticipatory behavior, producing a satiety signal. Insulin signaling in the nucleus accumbens produces a strong reward signal by amplifying action-potential-dependent dopamine release through activation of cholinergic interneurons that express insulin receptors. Thus, at least in the case of eating behavior, the role of insulin is complex, inducing both satiety and reward signals. The net effect of CNS insulin during overnight abstinence from smoking is a rapid disruption of nicotine reward.

The data reported herein show that intranasal insulin restores blunted cortisol response to stress brought upon by acute two-night withdrawal from nicotine. Rapid reversal of blunted response to stress with insulin occurs in streptozotocin-diabetic rats, indicating that plastic processes resulting in an impaired capacity to stimulate the hypothalamic-pituitary adrenal axis can, in fact, be acutely reversed. This phenomenon is independent of hypoglycemia. In a hyperinsulimic-euglycemic clamp study—where peripheral levels of glucose are held constant while insulin is increased gradually—the significant rise in corticosterone levels was coupled with increased glucocorticoid receptor mRNA in the anterior pituitary and paraventricular nucleus, a mechanism that may be involved in the finding that central insulin reverses nicotine-adapted allostatic load.

In this study, a minor decline in plasma glucose concentrations was observed. Independent of its direct peripheral action on controlling glucose, insulin in the CNS also acts indirectly to regulate glucose metabolism. Central insulin action reduces hepatic glucose production by suppressing vagal activation via the nicotinic alpha 7 acetylcholine receptor. The slight drop in glucose may be due to this or another central mechanism and is unlikely due to peripheral insulin because blood tests revealed no significant changes in circulating insulin.

Intranasal insulin, formulated as previously described (Claxton et al., 2015. J Alzheimers Dis 45(4):1269-1270; Craft S. 2012. Nat Rev Neurol 8(7):360-362), produced a notable burning sensation. Commercially available injectable insulin contains, among other ingredients, zinc, which is a nasal irritant.

Intranasal route of administration to humans increases CSF insulin levels without changing peripheral insulin levels. General consensus exists that the route of intranasal insulin entry is in the olfactory region via olfactory nerves, although a small contribution of trigeminal nerve contribution (innervating both olfactory and respiratory regions) has not been ruled out. If a molecule can be transported across the olfactory region into CSF, the speed by which it reaches the fluid provides information regarding the mechanism by which it is transported across the olfactory epithelium. As insulin rapidly (i.e., within minutes) reaches the CSF, it likely does so via extracellular mechanisms, as intracellular processes take longer to reach CSF. Some have, however, questioned the ability of molecules to move extracellularly between tight junctions of olfactory sensory neurons and sustentacular cells. Radiolabeled insulin administered intranasally an animal studies is taken up into the olfactory bulb, hypothalamus, hippocampus, cerebellum, and whole brain within 2.5 minutes and is still present 60 minutes from the time of intranasal administration (Salameh et al., 2015. Journal of Alzheimer's Disease 47(3):715-728).

Thus, this disclosure describes compositions and methods for treating acute nicotine withdrawal after cessation of smoking for up to 36 hours. Generally, the method includes intranasally administering to the subject a dose of insulin effective to ameliorate at least one symptom or clinical sign of acute nicotine withdrawal.

Thus, this disclosure describes compositions and methods for treating nicotine withdrawal associated with cessation of smoking. Generally, the method includes intranasally administering to the subject a dose of insulin effective to ameliorate at least one symptom or clinical sign of nicotine withdrawal. As used herein, “treat” or variations thereof refer to reducing, limiting progression, ameliorating, or resolving, to any extent, the symptoms or signs related to acute nicotine withdrawal after cessation from smoking for up to 36 hours). A “treatment” may be therapeutic or prophylactic. As used herein, “therapeutic” and variations thereof refer to a treatment that ameliorates one or more existing symptoms or clinical signs associated with acute nicotine withdrawal. As used herein, “prophylactic” and variations thereof refer to a treatment that limits, to any extent, the development and/or appearance of a symptom or clinical sign of acute nicotine withdrawal. Generally, a “therapeutic” treatment is initiated after a symptom or clinical sign of acute nicotine withdrawal manifests in a subject, while “prophylactic” treatment is initiated before a symptom or clinical sign of acute nicotine withdrawal manifests in a subject. Also as used herein, “symptom” refers to any subjective evidence of disease or of a patient's condition, while “sign” or “clinical sign” refers to an objective physical finding relating to a particular condition capable of being found by one other than the patient.

The insulin may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the insulin without causing any undesirable biological effects or interacting in a deleterious manner with any of the other components of the pharmaceutical composition in which it is contained.

The insulin may therefore be formulated into a pharmaceutical composition. The pharmaceutical composition may be formulated in a variety of forms adapted to a preferred route of administration. Thus, a composition can be administered via known routes including, for example, intranasally. A pharmaceutical composition can be administered to a mucosal surface, such as by administration to, for example, the nasal or respiratory mucosa (e.g., by spray or aerosol).

Thus, the insulin may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, an aerosol formulation or a non-aerosol spray. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the insulin into association with a carrier that constitutes one or more accessory ingredients. In general, a formulation may be prepared by uniformly and/or intimately bringing the active compound into association with a liquid carrier, a finely divided solid carrier, or both.

The amount of insulin administered can vary depending on various factors including, but not limited to, the weight, physical condition, and/or age of the subject, and/or the route of administration. Thus, the absolute amount of insulin included in a given unit dosage form can vary widely, and depends upon factors such as the species, age, weight and physical condition of the subject, and/or the method of administration. Accordingly, it is not practical to set forth generally the amount that constitutes an amount of insulin effective for all possible applications. Those of ordinary skill in the art, however, can readily determine the appropriate amount with due consideration of such factors.

In some embodiments, the method can include administering sufficient insulin to provide a minimum dose of at least 25 IU such as, for example, at least 45 IU, at least 50 IU, at least 55 IU, at least 60 IU, or at least 65 IU. In some embodiments, the method can include administering sufficient insulin to provide a maximum dose of no more than 75 IU such as, for example, no more than 70 IU, no more than 65 IU, no more than 60 IU<no more than 55 IU<, or no more than 50 IU. In some embodiments, the method can include administering sufficient insulin to provide a dose within a range having as endpoints any minimum does listed above and any maximum dose listed above that is greater than the minimum dose. Thus, in certain embodiments, the method can include administering sufficient insulin to provide a dose of from 45 IU to 70 IU such as, for example, from 55 IU to 65 IU. In one particular embodiment, the method includes administering sufficient insulin to provide a dose of 60 IU.

In some embodiments, insulin may be administered, for example, from a single dose to multiple doses per week, although in some embodiments the method can be performed by administering insulin at a frequency outside this range. In certain embodiments, the insulin may be administered from about once per month to about five times per week.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Participants

Men and women aged 18 to 65 and smoking 10 or more cigarettes per day for the past year were recruited from the community via local advertisements and were assessed for possible study participation. Their smoking status was verified by carbon monoxide concentration greater than 6 ppm¹¹.

Participants were eligible to participate if they had normal vital signs (blood pressure<140/90 mmHg; heart rate between 50-100 beats per minute, body temperature<38° C.); point-of-care blood glucose between 80-140 mg/dl; and body mass index between 18.5-30 kg/m² (measured by InBody230 Bioelectrical Impedance Analysis). Only participants with a urine sample negative for psychotropic substances (amphetamine, barbiturates, benzodiazepines, cocaine, ecstasy, marijuana, methadone, methamphetamine, opiates, phencyclidine, and tricyclic antidepressants) were allowed to participate. Additionally, subjects had to score ≥80 on Shipley IQ (vocabulary standard score) to qualify for the study.

Individuals positive for any DSM IV-R Axis I disorder were disqualified from participating in the study. Hyposmic or anosmic individuals (e.g., those who identified fewer than 10 of 12 smells correctly) were excluded, as well as individuals who were currently using any intranasal medications or who had structural/functional abnormalities of their nasal airway identified during a physical exam. Additional criteria for exclusion was a score >5 on the Michigan Alcoholism Screening Test¹², a Breath Alcohol Concentration >0.00% on an breathalyzer (Alco-Sensor FST, Intoximeters, Inc., St. Louis, Mo.), any current prescription or over-the-counter medication use, current pregnancy or lactation, current use of a smoking cessation aid, previous or current use of insulin, and any lifetime history of an endocrine disease or eating disorder.

The United States Food and Drug Administration approved the Investigational New Drug application (#116626), and the study was approved by the University of New Mexico Human Research Review Committee. Written consent was obtained from all participants, and participants were compensated for their time.

### Procedures

This was a two-session study, single blind study with Session 1 evaluating efficacy of intranasal insulin on various cognitive measures. Session 2 methodology and results are presented in this publication.

Subjects started their overnight abstinence at 8 PM the night before reporting to the University of New Mexico Clinical and Translational Science Center. At 8 AM the following day, abstinence from smoking was confirmed with a CO reading on a coVita piCO+ breathalyzer less than 50% of their baseline CO recorded at intake. A subset of individuals (N=19; 52% of the sample) had a serum analysis verified nicotine level. Exposure to nicotine was analyzed by quantitative liquid chromatography-tandem mass spectrometry. Cutoff for nicotine was 2 ng/mL for nicotine.

None of the tested subjects had a detectable nicotine level, meeting the study's abstinence criteria (i.e., less than 50% of baseline CO level). Following negative urine drug and pregnancy tests, at 8:30 AM, subjects completed their morning assessment of smoking urges and mood. A salivary cortisol sample was also taken at this time. They were then provided breakfast (a slice of banana bread, one apple, and two cheese sticks), which they had to eat entirely. The rest of the morning, subjects watched and read emotionally neutral movies and literature. At 11:45 AM, a study nurse inserted the intravenous line, completed a point-of-care (POC) glucose check and nares assessment. If a subject was found to have a glucose reading <70 mg/dL or to have any nasal pathology, that subject was not allowed to proceed with the study.

Intranasal spray administration of insulin or placebo occurred at 12:30 PM. The purpose of the spacing time between morning check-in and nasal spray administration was stabilization of postprandial insulin levels. In a randomized fashion, the study nurse administered one spray in each nostril every three minutes for the total of six sprays of either insulin or placebo. Instructions to perform the Trier Social Stress Test were given at 13:20, and speech delivery occurred 13:30. Briefly, the Trier Social Stress Test¹³ is a standardized laboratory stressor consisting of a free speech and a mental arithmetic task in front of an audience. Participants were introduced to the task and instructed to prepare a 5-minute presentation in which they were to promote their candidacy for a job. The speech was delivered after a 10-minute preparation period. Thereafter, participants were introduced to the mental arithmetic task, also standing in front of the audience and a self-projecting television screen. Subjects were required to count backwards from 2023 in steps of 17 as fast and accurately as possible for five minutes; upon making a mistake, subjects were asked to stop and start again at 2023. At every post-stress timepoint (13:40, 13:50, 14:00, 14:10, and 14:40), study nurses checked POC glucose to ensure euglycemia. After the last time point at 14:40, subjects ate a snack, had their glucose level checked, received another nares exam, and were discharged from the clinic at 16:00.

### Laboratory Assessments

Subjective measures included the Questionnaire on Smoking Urges (QSU) Brief Form and Positive and Negative Affect Schedule (PANAS). Urges to smoke were rated using the 10-item QSU Brief Form¹⁴ that consists of 10-questions. The answers are scored from 1 (strongly disagree) to 7 (strongly agree). These questions are the basis for the two factor derived subscales. Factor 1 subscale reflects a strong desire and intention to smoke with smoking perceived as rewarding. Factor 2 subscale reflects an anticipation of relief from negative affect with an urgent desire to smoke. Study data analysis included evaluations of both factors. The PANAS (Watson et al., 1988. J Pers Soc Psychol 54(6):1063-1070) consists of 10 negative and 10 positive mood terms rated on five-point Likert-type response scales from 1 (very slightly or not at all) to 5 (extremely). Positive and negative scale scores each range between 10 and 50. The PANAS was chosen as a composite measure of self-reported positive and negative affect. Subjective measures were taken at 08:30, 12:20, 13:00, 13:40, 13:50, 14:00, 14:10 and 14:40.

Salivary measures: Saliva samples were collected using a saliva collection kit (Salimetrics, LLC, Carlsbad, Calif.). Saliva samples were collected at 08:30, 12:20, 13:00, 13:40, 13:50, 14:00, 14:10, and 14:40, and analyzed for cortisol as follows: A microtitre plate was coated with monoclonal antibodies to cortisol using salivary cortisol ELISA kit (Salimetrics, LLC, Carlsbad, Calif.). After incubation, unbound components were washed away. Bound cortisol peroxidase was measured by the reaction of the peroxidase enzyme on the substrate tetramethylbenzidine (TMB). This reaction produced a blue color. A yellow color was formed after stopping the reaction with sulfuric acid. Optical density was read on a standard plate at 450 nm.

Cardiovascular measures: A continuous heart rate monitor (BIOHARNESS 3, Zephyr Technology, Annapolis, Md.), measuring heart rate every second, was placed at 12:00 and was kept on until 14:40. Blood pressure was collected at 12:20, 13:00, 13:40, 13:50, 14:00, 14:10, and 14:40.

Blood measurements: Blood samples were analyzed for measurements of insulin, glucose, C-peptide, and non-esterified fatty acids.

Serum insulin was analyzed on the Immulite 1000 immunoassay system (Siemens Healthcare GmbH, Erlangen, Germany). This is a solid-phase, enzyme-labeled chemiluminescent immunometric assay. The solid phase (bead) was coated with monoclonal murine anti-insulin antibody. The liquid phase consisted of alkaline phosphatase (bovine calf intestine) conjugated to polyclonal sheep anti-insulin antibody and alkaline phosphatase (bovine calf intestine) conjugated to monoclonal murine anti-insulin antibody. The patient sample and the reagent were incubated together with the coated bead for 60 minutes. During this time, insulin in the sample forms the antibody sandwich complex with the monoclonal murine anti-insulin antibody on the bead, enzyme conjugated polyclonal sheep anti-insulin antibody, and enzyme conjugated monoclonal murine anti-insulin antibody in the reagent. Unbound patient sample and enzyme conjugate were then removed by centrifugal washes. Finally, chemiluminescent substrate was added to the test unit containing the bead and the signal is generated in proportion to the bound enzyme.

Serum glucose was performed on the ACE ALERA system (Alfa Wassermann Diagnostic Technologies, S.p.A., Bologna, Italy). An increase in absorbance of NADH is directly proportional to glucose concentration, and was measured bichromatically at 340 nm/378 nm. NAD+ does not absorb strongly at this wavelength.

Serum C-peptide was performed on the Immulite 1000 immunoassay system (Siemens Healthcare GmbH, Erlangen, Germany). This is a solid-phase, two-site chemiluminescent immunometric assay. The solid phase (bead) is coated with monoclonal murine anti-C-peptide antibody. The liquid phase consists of alkaline phosphatase (bovine calf intestine) conjugated to monoclonal murine anti-C-peptide antibody in buffer. The patient sample and the reagent were incubated together with the coated bead for 30 minutes. During this time, C-peptide in the sample forms the antibody sandwich complex with monoclonal murine anti-C-peptide antibody in the reagent. Unbound patient sample and enzyme conjugate were then removed by centrifugal washes. Finally, chemiluminescent substrate was added to the Test Unit containing the bead and the signal is generated in proportion to the bound enzyme.

Blood samples were collected at 12:20, 13:00, 13:40, and 14:10 and analyzed for non-esterified fatty acids using an AU 480 chemistry analyzer (Beckman Coulter, Inc., Brea, Calif.) using a NEFA-HR(2) kit (Wako Chemicals USA, Inc., Cape Charles, Va.).

### Drug Preparation

The drug substance used for preparation of Intranasal Insulin was bulk human Regular insulin (U100 Novolin R, Novo Nordisk US). Placebo contained normal saline (0.9% NaCl). The product was prepared in a biosafety cabinet 30 minutes before administration. Insulin or placebo was placed in a 10 mL amber glass bottle (Gerresheimer AG, Düsseldorf, Germany), which was capped with a 10 mg sterile nasal pump (Aero Pump GmbH, Hochheim, Germany). The assembled product was primed per manufacturer's recommendations before 0.6 ml volume was administered to study participants. Each spray delivers 0.1 ml, and since the concentration of insulin is 100 IU/ml, six sprays delivered 60 IU of insulin.

### Data Analysis

Random coefficient models were constructed for analyzing subjective (i.e., craving and mood), blood (insulin, glucose, C-peptide and nonesterified fatty acids) and cardiovascular (blood pressure) measures. For every model, an evaluation of non-linear term was performed, and, if significance was detected, that polynomial term for time was included in the model. Treatment, time and treatment by time interaction were included as fixed effects. Time nested within subjects was included as a random effect. If an interaction was detected, post hoc comparisons were performed comparing treatment groups at each time point. Cortisol was log-transformed for the purpose of normalizing distribution. The summary measure for cortisol analysis was peak change from baseline. Baseline cortisol was the 8:30 AM measurement, and time period for determination of peak was between 13:40 and 14:40. This time period reflects any cortisol change that resulted from the psychosocial stress procedure, with speech occurring from 13:30 to 13:35 and math from 13:35 to 13:40. Group differences were compared using analysis of variance (ANOVA). Significance values are reported as detected; no adjustments were made for multiple comparisons.

To accurately summarize a large amount of heart rate data (measured every second), eight summary measures were calculated: baseline, instructions, speech, math and four recovery time points. Baseline was computed as 10-minute averages (12:50 to 13:00). The first minute of instructions, speech and math were the summary measures of those particular components of the task. Recovery points were computed as 10-minute averages starting at 13:40, 13:50, 14:00, and 14:10.

Group differences in subject characteristics were evaluated using t-test and chi-square statistics; morning subjective and cortisol measures were evaluated using t-test statistic.

In the nicotine craving analysis, one subject's data was dropped from the analysis because the subject reported mistaking the rating scale as low when it was actually high. It could not be determined when the subject switched to the correct intensity rating, hence, this subject's results were not included in the analysis.

### Example 2

In summary, the purpose of this inpatient study was to evaluate efficacy of intranasal insulin on snack intake during Day 1 of smoking abstinence and nicotine cravings on Day 2 of smoking abstinence. Results from Day 2 are presented. The main differences between Example 1 and Example 2 are: (1) parallel vs crossover design, and (2) placebo content: the parallel design placebo was normal saline, the crossover design was 8.7% NaCl. The purpose of increasing the placebo salt content in the crossover study was to maintain the blind.

### Participants

Men and women (aged between 18 and 65) smoking 10 or more cigarettes for the past year recruited from the community via local advertisements were evaluated for study participation. Their smoking status was verified by carbon monoxide concentration greater than 6 ppm (Middleton E T, Morice A H, 2000. Chest 117(3):758-763).

Subjects were eligible to participate if they had normal vitals (blood pressure<140/90; heart rate between 50-100, body temperature<37.4° C.); point-of-care blood glucose between 80-140; and body mass index between 18.5-30 kg/m² (measured by InBody230 Bioelectrical Impedance Analysis). Only subjects with a urine sample negative for psychotropic substances (amphetamine, barbiturates, benzodiazepines, cocaine, ecstasy, marijuana, methadone, methamphetamine, opiates, phencyclidine, tricyclic antidepressants) were eligible to participate. Additionally, subjects had to score ≥80 on Shipley IQ (vocabulary standard score) and have a normal score on eating disinhibition and restraint tests (Salameh et al., 2015. J Alzheimers Dis 2015. 47(3):715-728).

Individuals positive for any DSM IV-R Axis I disorder were disqualified from participating in the study. Hyposmic or anosmic individuals (identifying less than 10 of 12 total smells correctly) were excluded as well as individuals who currently use any intranasal medications. Subjects with structural/functional abnormalities identified during a physical exam were excluded from the study. Additional criteria for exclusion included Michigan Alcoholism Screening Test score >5 (Selzer M L, 1971. Am J Psychiatry 127(12):1653-1658), Breath Alcohol Concentration >0.00% on a breathalyzer (Alco-Sensor FST, Intoximeters, Inc., St. Louis, Mo.), current prescription or over the counter medication use, current pregnancy or lactation, current use (i.e., within the past three months) of a smoking cessation aid, previous/current use of insulin and lifetime history of endocrine disease or eating disorder.

### Procedures

This was a proof-of-concept, randomized crossover single blind Phase II trial comparing efficacy of intranasal insulin (60 IU) versus placebo in decreasing snacking and smoking urges.

Study participants began their first night of smoking abstinence at 20:00 prior to reporting to the inpatient unit of University of New Mexico Clinical and Translational Science Center at 11:30 the following day. During Day 1 at the hospital, participants provided a CO sample, which had to be 50% of their measurement at screening or less than 7 ppm and a negative breath alcohol test (0.00%). Additional criteria for session continuation included negative urine drug test and vitals within normal limits as described in the “Participants” section above. The study nurse evaluated the nares for abnormalities and checked POC glucose to ensure subjects were within normal limits prior to receiving spray.

During Day 1, participants were administered spray at 12:40, ate lunch at 13:00 and completed appetite ratings and a snacking test. Participants ate dinner at 19:00 and lights were turned off at 21:00.

The following morning, participants completed Questionnaire of Smoking Urges at 7:50. Prior to this first questionnaire administration, participants provided a urine sample, which had to be negative for illicit substances, and a CO sample, which had to be either 50% lower than the value on day 1, or less than 7 ppm. An intravenous line was established at 6:45 and a baseline blood sample was drawn. Study nurse checked POC glucose and completed a nares evaluation to confirm participant was within normal limits to receive spray. Spray administration occurred at 8:00, followed by breakfast at 8:20. Post-spray Questionnaire of Smoking Urges was completed at 8:15, 8:45, 9:15, 9:45, 10:30 and 11:30. During this time blood samples were taken for additional analyses. At noon, study nurse evaluated the nares to rule out abnormality and checked POC glucose to ensure that results were within normal limits. The two sessions were separated by one week.

### Outcomes

Urges to smoke were rated using the 10-item Questionnaire on Smoking Urges-Brief (Cox et al., 2001. Nicotine Tob Res 3(1):7-16) that consists of 10-questions. The answers are scored from 1 (strongly disagree) to 7 (strongly agree). These questions are the bases for the two factor derived subscales. Factor 1 subscale reflects a strong desire and intention to smoke with smoking perceived as rewarding. Factor 2 subscale reflects an anticipation of relief from negative affect with an urgent desire to smoke. These two subscales were sub-analyses stemming from our main (overall) QSU-Brief analysis. Cravings were measured at −15, 0, +20, +60, +70, +80, +90 and +120 minutes from spray administration on Day 2.

Additional outcome measures included safety measures, i.e., post-spray symptom-specific subjective ratings and adverse events. Post-spray symptom-specific subjective ratings (0-10) of burn, pain, cough, and taste were collected after each spray round. Administration instructions were one spray in each nostril every three minutes for the total of six sprays. Hence, the data reflect subjective measures listed above after each of the total of three rounds of spray administration. Adverse events were recorded and collected following the completion of every session as well as completion of the entire study.

### Drug Preparation and Administration

The drug substance used for preparation of Intranasal Insulin was bulk Novolin R (Novo Nordisk US). Placebo contained 8.7% NaCl. Per FDA approved protocol, the product was prepared in a biosafety cabinet of UNM Translational Radiopharmacy. Drug Preparation occurred 30 minutes before administration. Inulin or placebo was placed in a 10 mL amber glass bottle (Gerresheimer AG, Düsseldorf, Germany), which was capped with a 10 mg sterile nasal pump (Aero Pump GmbH, Hochheim, Germany). Assembled product was primed per manufacturer's recommendations before 0.6 ml volume was administered to study participants. In a randomized fashion, study nurse administered one spray in each nostril every three minutes for the total of six sprays of either insulin or placebo. As each spray delivers 0.1 ml of fluid, and the concentration of insulin in Novolin R is 100 IU/mL, administering six sprays delivers 60 IU of insulin.

### Data Analysis

Random coefficient models were constructed for analyzing study outcomes. Treatment, time, treatment by time interaction, and sequence were included as fixed effects. Time nested within subjects, and time nested within treatment nested within subjects were included as random effects. Adverse events are presented in a descriptive fashion. No adjustment for multiple tests was made; all p values are reported at their nominal level.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method of treating a subject in need of treatment for acute nicotine withdrawal, the method comprising:
intranasally administering to the subject a dose of insulin effective to ameliorate at least one symptom or clinical sign of nicotine withdrawal.

2. The method of claim 1 wherein the dose of insulin comprises at least 45 IU and no more than 75 IU.

3. The method of claim 2 wherein the dose comprises 60 IU.

4. The method of claim 1 wherein the dose is an amount effective to restore at least a portion of decreased cortisol level in saliva following a stress stimulus, the decreased cortisol level being associated with acute nicotine withdrawal.

5. The method of claim 1 wherein the dose is an amount effective to decrease an urge to smoke a cigarette at the time of acute withdrawal from nicotine.
